(19) United States
(12) Patent Application Publication
(10) Pub. No.: US 2025/0254472 A1
(43) Pub. Date: Aug. 7, 2025

(54) WIRELESS STREAMING FROM MULTIPLE SOURCES FOR AN IMPLANTABLE MEDICAL DEVICE

(71) Applicant: Cochlear Limited, Macquarie University, NSW (AU)

(72) Inventors: Michael Goorevich, Naremburn, NSW (AU); Phyu Phyu Khing, North Sydney, NSW (AU); Michael John Phillips, Alfords Point, NSW (AU)

(21) Appl. No.: 18/855,919

(22) PCT Filed: Apr. 12, 2023
(86) PCT No.: PCT/IB2023/053752

§ 371 Date: Oct. 10, 2024

Related U.S. Application Data
(60) Provisional application No. 63/332,558, filed on Apr. 19, 2022.

(51) Int. Cl. H04R 25/00; A61N 1/36; A61N 1/372

(52) U.S. Cl. CPC H04R 25/43; A61N 1/36038; A61N 1/37223; H04R 25/554; H04R 2225/67

(57) ABSTRACT

Presented herein are techniques for receiving a plurality of streams by an implantable component of an implantable medical device system from a plurality of wireless sources and mixing the plurality of streams to generate stimulation signals for use in stimulating a recipient of the implantable medical device system.

### BACKGROUND

### Field of the Invention

The present invention relates generally to implantable medical devices in which multiple wireless streams are provided for an implantable medical device.

### Related Art

Medical devices have provided a wide range of therapeutic benefits to recipients over recent decades. Medical devices can include implantable components/devices, external or wearable components/devices, or combinations thereof (e.g., a device having an external component communicating with an implantable component). Medical devices, such as traditional hearing aids, partially or fully-implantable hearing devices (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a first method is provided. The first method comprises: receiving a plurality of wireless streams by an implantable component of an implantable medical device system from a plurality of wireless sources; and mixing the plurality of wireless streams to generate stimulation signals for use in stimulating a recipient of the implantable medical device system.

In another aspect, one or more non-transitory computer readable storage media are provided. The one or more non-transitory computer readable storage media comprise instructions that, when executed by a processor, cause the processor to: receive a plurality of wireless streams by an implantable component of an implantable medical device system from a plurality of wireless sources; and mix the plurality of wireless streams to generate stimulation signals for use in stimulating a recipient of the implantable medical device system.

In another aspect, an implantable component of an implantable medical device system is provided. The implantable component comprises memory for storing data and one or more processors, wherein the one or more processors are configured to: receive a plurality of wireless streams from a plurality of wireless sources; and mix the plurality of wireless streams to generate stimulation signals for use in stimulating a recipient of the implantable medical device system.

In another aspect, a second method is provided. The second method comprises: receiving, from a first external device, a first indication of a first signal for use by the implantable medical device to generate stimulation signals for delivery to a recipient of the implantable medical device; receiving, from a second external device, a second indication of a second signal for use by the implantable medical device to generate stimulation signals for delivery to the recipient; contemporaneously receiving the first signal from the first external device and the second signal from the second external device; and generating stimulation signals for delivery to the recipient based on the first signal and the second signal.

In another aspect, one or more non-transitory computer readable storage media are provided. The one or more non-transitory computer readable storage media comprise instructions that, when executed by a processor, cause the processor to: receive, from a first external device, a first indication of a first signal for use by the implantable medical device in stimulating a recipient of the implantable medical device; receive, from a second external device, a second indication of a second signal for use by the implantable medical device in stimulating the recipient; contemporaneously receive the first signal from the first external device and the second signal from the second external device; and stimulate the recipient using the first signal and the second signal.

In another aspect, an implantable medical device is provided. The implantable medical device comprises memory for storing data and one or more processors, wherein the one or more processors are configured to: receive, from a first external device, a first indication of a first signal for use by the implantable medical device in stimulating a recipient of the implantable medical device; receive, from a second external device, a second indication of a second signal for use by the implantable medical device in stimulating the recipient; contemporaneously receive the first signal from the first external device and the second signal from the second external device; and stimulate the recipient using the first signal and the second signal.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIG. 2 is a functional block diagram illustrating further details of an example audio signal processing path of a cochlear implant system configured to implement certain techniques presented herein;

FIG. 3 is a functional block diagram illustrating details of an example use case for an implantable component configured to implement certain techniques presented herein;

FIG. 4 is a schematic diagram of an example packet that can be received from a streaming source by an implantable component, according to certain embodiments;

FIG. 5 is a functional block diagram illustrating further details of another example use case for an implantable component configured to implement certain techniques presented herein;

FIG. 6 is a functional block diagram illustrating further details of another example use case for implantable component configured to implement certain techniques presented herein;

FIG. 7 is a functional block diagram illustrating further details of another example use case for an implantable component configured to implement certain techniques presented herein;

FIG. 8 is a functional block diagram illustrating further details of another example use case for an implantable component configured to implement certain techniques presented herein;

FIG. 9 is a functional block diagram illustrating further details of another example use case for an implantable component configured to implement certain techniques presented herein;

FIG. 10 is a functional block diagram illustrating further details of another example use case for an implantable component configured to implement certain techniques presented herein;

FIG. 11 is a flowchart illustrating an example process to facilitate wireless streaming from multiple sources for an implantable component of a medical device system according to certain embodiments;

FIG. 12 is a flowchart illustrating another example process to facilitate wireless streaming from multiple sources for an implantable component of a medical device system according to certain embodiments; and

FIG. 13 is a functional block diagram of an implantable stimulator system with which aspects of the techniques presented herein can be implemented.

### DETAILED DESCRIPTION

Certain implantable medical device systems, such as implantable auditory prostheses, include both an implantable component and an external component. For example, when a medical device is embodied as a hearing device, such as a cochlear implant system or other auditory prosthesis provided for a recipient, environmental signals, such as sensory or sound signals, also referred to herein as audio signals, are typically captured or received by an external component. The external component is typically configured to process the sensory/sound signals via sound processing logic and provide signal data (e.g., sensory signal data, such as audio signal data or stimulation signal data) to an implantable component, typically referred to as a cochlear implant or, more generally the “implant,” for the cochlear implant system in which the implantable component facilitates delivery of electrical stimulation (current) to the recipient's cochlea.

Current cochlear implant systems are typically implemented using a well-defined and proprietary data interface between the external component and the implantable component, which typically meets the criteria of the implant design (e.g., the implant design drives the criteria for the interface data/signals communicated between the external component and the internal component). For example, the implant can expect a continuous sequence of commands of some format that define stimulation at one or more electrodes, or “channels,” which, thus, defines the interface for communicating data (audio data or stimulation data) between the sound processing logic of the external component and the implant.

One limitation with such an approach is that the combination of sound processing logic and the implant are typically “designed for each other,” which is sensible given the potential safety concerns of an implantable device provided for a recipient. However, if an implant was able to process a direct audio/sensory signal received at the implant, also referred to herein as a “stream,” then any number of audio signal streams (and/or or encrypted/encoded/compressed audio signal streams) could be directly received from external/remote devices. An audio signal is a safe, well understood and common signal type. Further, such an enhancement could also be extended to an implantable device being capable of receiving any number of (proprietary) stimulation protocol signal streams, if the device were capable of processing multiple stimulation signal streams received from multiple sources. As referred to herein a “stream” can include any form of data (audio signal data, stimulation signal data, etc.) that can be transmitted in a continuous or semi-continuous flow of packets/frames. In various embodiments, streams can be transmitted wirelessly by a wireless source over one or more wireless connections/interfaces (e.g., a “wireless stream,” a “wireless packet stream,” “a wireless frame stream” a “wireless signal stream,” etc.”), by a wired source, and/or can be generated by one or more components within a medical device system, such as any combination of an external component and/or an internal component of the medical device system (e.g., an “internal stream,” etc.).

Presented herein are techniques through which various implant interfaces can be defined that allow any combination of audio signal streams and/or stimulation signal streams to be contemporaneously received by an implantable component of a cochlear implant system from any combination of devices, also referred to herein as remote devices, external device, or sources, that are external to the implant, such that the management of streams received from multiple sources can be processed by the implant. Further, in some embodiments audio/stimulation signal streams received from one or more remote sources can be managed along with any combination of sensory/sound/audio data/signals received/processed by the implant (via one or more sound/input devices and sound processing logic provided for the implant), stimulation/audio signals/data received from an external component of a cochlear implant system, and/or stimulation/audio signals generated by sound processing logic of the implant itself.

One potential benefit of the techniques presented herein is that an implant can be simultaneously compatible with a wide range of possible sound sources, from traditional sound processors (e.g., sending stimulation data streams) to wireless microphones and smartphones (e.g., sending audio data streams).

Broadly, techniques herein provide that an implantable component (implant) of a medical device system, such as a cochlear implant system, can manage multiple streams (received from multiple sources and/or generated by the implant itself) in order to provide stimulation to a recipient. For example, the implant can be listening to or “paired” or otherwise wirelessly linked (e.g., via a Bluetooth® Low Energy (BLE) pairing, via an Institute of Electrical and Electronics Engineers (IEEE) 802.11 (Wi-Fi®) connection, or the like) with a number of different potential wireless sources, which can include any combination of proprietary stimulation protocol streaming sources and/or or audio streaming sources.

In some instances, one or more wireless source(s) can indicate to the implant that a signal is to be presented to the recipient based on a stream provided by the source(s), as well as indicating what type of source (stream) is being provided (e.g., proprietary stimulation protocol signal stream or audio signal stream). For instances involving a proprietary stimulation protocol signal stream being provided to an implant, a source can indicate the protocol type and version so that the implant can correctly decode the stream. In some instances, for audio sources, a remote source can indicate an encoding type and version for a given audio signal stream so that the implant can appropriately decode/decrypt/decompress/etc. the audio signal stream.

In some instances, indicating the type of encoding for an audio signal stream may not be needed if implied by the source type from which the stream is obtained. In still some instances, an implant can determine which sources to use and which to ignore for generating stimulation for a recipient. For example, in some instances this decision could be based on recipient (user) preference and/or any other information such as location, time of day, etc. Other variations can be envisioned, as discussed in further detail herein, below.

Once the implant has determined which sources to “listen” to (e.g., the streams from which sources to process and which to ignore), the implant can determine how to combine the streams received from these sources. For example, different proprietary stimulation protocol signal streams including various channel amplitudes could have amplitudes summed together, effectively “mixing” the sound sources. Similarly, audio sources, also referred to herein as sound sources, such as any combination of audio streaming sources and/or audio signal source (e.g., received/captured via sound sensors configured for an implant) can be decoded and summed to combine them together, such as, for example, audio source from a television as well as from a public address system. In some instances, additional processing can be added to signals received from sound sources prior to summing, such as a mixing ratio or frequency adjustment, compression, latency compensation, etc. The implant can further combine any combination proprietary stimulation protocol signal streams with the signals obtained from any combination of audio/sound sources appropriately, in order to combine all source types for presentation to the user.

In some instances, multiple streaming sources and source types could also be sent to an implant, simultaneously, over the same wireless link in different channels of the same wireless system, such as 2.4/5 GHz Wi-Fi, BLE, or other wireless communication protocol. For example, a simultaneous audio and stimulation encoded streaming data source could be transmitted by a wireless source from multiple inputs or signal paths of the wireless source. During operation, an implant can continuously monitor an environment for new and/or changed source streams and/or source/stream configurations to update stimulation provided to a recipient.

Merely for ease of description, the techniques presented herein are primarily described with reference to a specific medical device system, namely a cochlear implant system. However, it is to be appreciated that the techniques presented herein can also be partially or fully implemented by other types of implantable medical device systems. For example, the techniques presented herein can be implemented by other auditory prosthesis or hearing device systems, such as hearing aids, middle ear auditory prostheses, bone conduction devices, direct acoustic stimulators, electro-acoustic prostheses, auditory brain stimulators, combinations or variations thereof, etc. The techniques presented herein can also be implemented by dedicated tinnitus therapy devices and tinnitus therapy device systems. In further embodiments, the techniques presented herein can also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc. A cochlear implant system can be referred to interchangeably herein as an implantable hearing device system.

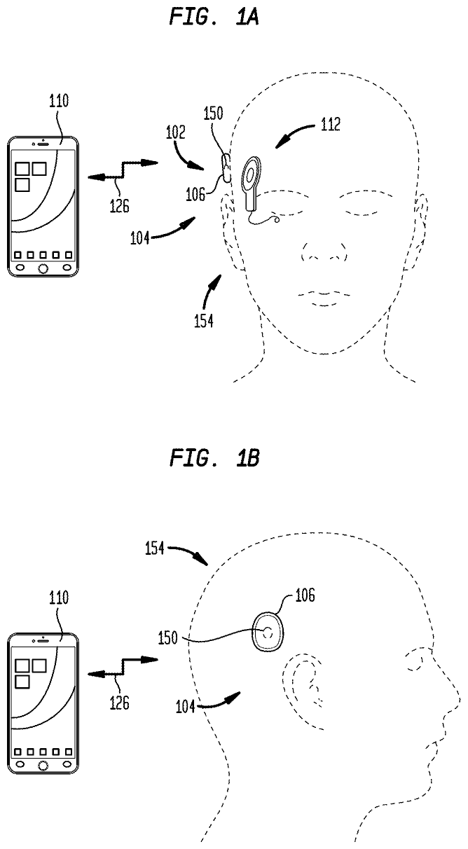

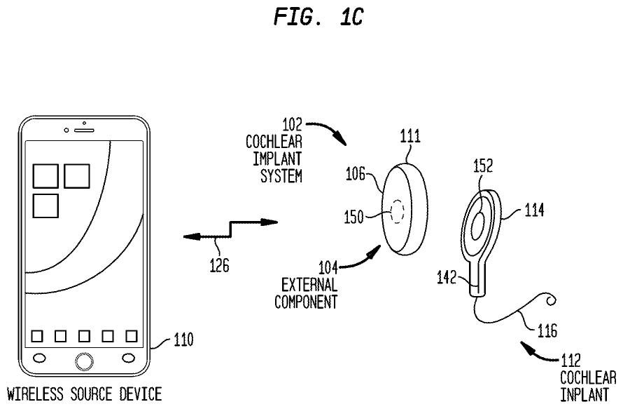

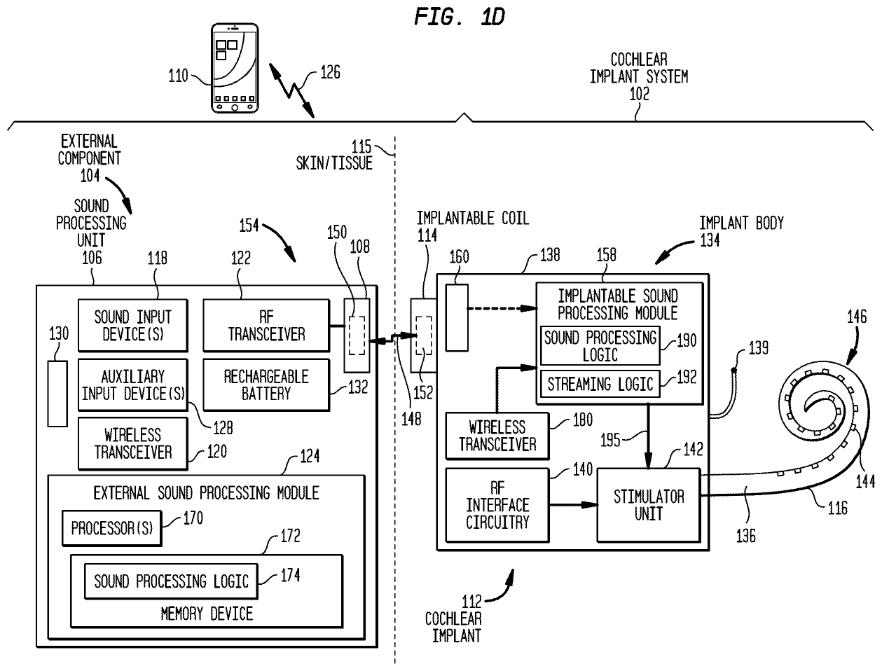

FIGS. 1A-1D illustrate an example hearing device system, in particular, a cochlear implant system 102 with which aspects of the techniques presented herein can be implemented. The cochlear implant system 102 comprises an external component 104 and an implantable component 112. In the examples of FIGS. 1A-1D, the implantable component 112 is sometimes referred to as a “cochlear implant,” “implantable component,” or, more generally, an “implant.” FIG. 1A illustrates the implantable component 112 implanted in the head 154 of a recipient, while FIG. 1B is a schematic drawing of the external component 104 worn on the head 154 of the recipient. FIG. 1C is another schematic view of the cochlear implant system 102, while FIG. 1D illustrates further details of the cochlear implant system 102. For ease of description, FIGS. 1A-1D will generally be described together.

Cochlear implant system 102 includes an external component 104 that is configured to be directly or indirectly attached to the body of the recipient and an implantable component 112 configured to be implanted in the recipient. In the examples of FIGS. 1A-1D, the external component 104 comprises an external sound processing unit 106, while the implantable component 112 includes an implantable coil 114, an implant body 134, and an elongate stimulating assembly 116 configured to be implanted in the recipient's cochlea.

In the example of FIGS. 1A-1D, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 111 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external (headpiece) coil 108 (as illustrated in FIG. 1D) that is configured to be inductively coupled to the implantable coil 114.

It is to be appreciated that the OTE sound processing unit 106 is merely illustrative of the external devices that could operate with implantable component 112. For example, in alternative examples, the external component can comprise a behind-the-ear (BTE) sound processing unit or a micro-BTE sound processing unit and a separate external coil assembly. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sensory or sound signals which are then used as the basis for delivering stimulation signals to the recipient. The cochlear implant 112 can also operate in a second general mode, sometimes referred as an “invisible hearing” mode, in which the sound processing unit 106 is unable to provide sensory or sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sensory or sound signals itself via implantable sound sensors and then uses those sensory/sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with a wireless source device 110, also referred to herein as a wireless source 110, remote device, or source, configured to implement aspects of the techniques presented. The wireless source 110 can be a computing device, such as a computer (e.g., laptop, desktop, tablet), a mobile phone, remote control unit, a wireless access point (e.g., a source streaming through a Wi-Fi or Bluetooth router/switch, mesh access point, etc. to cochlear implant system 102), combinations thereof, and/or the like. It is to be understood that any wireless source discussed for embodiments herein can be capable of wirelessly communicating (potentially bi-directionally) with any combination of an external component or a cochlear implant of a cochlear implant system sound processing unit 106 or the cochlear implant 112) wirelessly communicate via a bi-directional communication link 126. The bi-directional communication link 126 can comprise, for example, any combination of short-range wireless communications, such as any combination of a Bluetooth link, a BLE link, a Wi-Fi link, a proprietary link, or any other wireless communication protocol.

Returning to the example of FIG. 1A-FIG. 1D, the OTE sound processing unit 106 comprises one or more input devices 113 that are configured to receive input signals (e.g., sound or data signals). In one instance, the one or more input devices 113 can include one or more sound input devices 118 (e.g., one or more external microphones, audio input ports, telecoils, etc.).

According to the techniques of the present disclosure, sound input devices 118 can include two or more microphones or at least one directional microphone. Through such microphones, directionality of the microphones can be optimized, such as optimization on a horizontal plane defined by the microphones. Accordingly, a classic beamformer design can be used for optimization around a polar plot corresponding to the horizontal plane defined by the microphone(s).

In some instances, input devices 113 for the sound processing unit 106 can also include one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120. However, it is to be appreciated that one or more input devices 113 can include additional types of input devices and/or less input devices (e.g., one or more auxiliary input devices 128 could be omitted).

The sound processing unit 106 also comprises the external coil 108, a charging coil 130, a closely-coupled transmitter/receiver 122, sometimes referred to as or radio-frequency (RF) transceiver 122, at least one rechargeable battery 132, and an external sound processing module 124. The external sound processing module 124 can comprise, for example, one or more processors and a memory device (memory), such as such as processor(s) 170 (e.g., one or more Digital Signal Processors (DSPs), one or more microcontroller cores, one or more hardware processors, etc.) and a number of logic elements, such as sound processing logic 174 stored in a memory device 172. The memory device 172 can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors, microcontrollers, DSPs, and/or the like that execute instructions for logic stored in the memory device.

The implantable component 112 comprises an implant body (main module) 134, a lead region 136, and the intra-cochlear stimulating assembly 116, all configured to be implanted under the skin/tissue (tissue) 115 of the recipient. The implant body 134 generally comprises a hermetically-sealed housing 138 in which RF interface circuitry 140, a stimulator unit 142, and an implantable sound processing unit or module 158 are disposed. The implant body 134 further includes a wireless transceiver 180 that facilitate wireless communications for the implant 112. The implant body 134 also includes the internal/implantable coil 114 that is generally external to the housing 138, but which is connected to the RF interface circuitry 140 via a hermetic feedthrough (not shown in FIG. 1D). It is to be understood that any wireless transceiver as discussed for various embodiments herein (e.g., wireless transceiver 120, wireless transceiver 180, etc.) can perform wireless (e.g., RF) transmission and reception of wireless signals via antenna(s)/antenna array(s) and one or more baseband processor(s) (modem), which can perform baseband modulation and demodulation, etc. associated with wireless signals to enable wireless communications for any external and/or implantable component discussed for various embodiments herein.

As noted, stimulating assembly 116 is configured to be at least partially implanted in the recipient's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the recipient's cochlea.

Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 and the implantable coil 114. In certain examples, the closely-coupled wireless link 148 is an RF link. However, various other types of energy transfer, such as infrared (IR), electromagnetic, capacitive and inductive transfer, can be used to transfer the power and/or data from an external component to an implantable component and, as such, FIG. 1D illustrates only one example arrangement.

As noted above, sound processing unit 106 includes the external sound processing module 124. In at least one embodiment, the external sound processing module 124 is configured to convert received input signals (received at one or more of the input devices 113) into output signals for use in stimulating a first ear of a recipient (i.e., the external sound processing module 124 is configured to perform sound processing on input signals received at the sound processing unit 106). Stated differently, the one or more processor(s) 170 in the external sound processing module 124 are configured to execute sound processing logic 174 in memory 172 to convert the received input signals into output signals that represent electrical stimulation for delivery to the recipient.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component (cochlear implant) 112 and the sound processing operations (e.g., conversion of sounds to output signals) can be performed by a processor within the implantable component 112.

In one embodiment with reference to FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sensory/sound signals.

As detailed above, in the external hearing mode the implantable component 112 can, in at least one embodiment, receive processed sensory/sound signals from the sound processing unit 106. However, in the invisible hearing mode, the cochlear implant 112 is configured to capture and process sensory/sound signals for use in electrically stimulating the recipient's auditory nerve cells. In particular, in at least one embodiment as shown in FIG. 1D, the cochlear implant 112 includes a plurality of implantable sound sensors 160 and an implantable sound processing module 158. Similar to the external sound processing module 124, the implantable sound processing module 158 can comprise, for example, for example, one or more processors (not shown) and a memory device (not shown) that includes sound processing logic 190 and streaming logic 192. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, any combination of microprocessors, microcontrollers, etc. that that execute instructions for the logic stored in the memory device.

Conventionally, in the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a recipient (e.g., the processing module 158 is configured to perform sound processing operations). Stated differently, the one or more processors in implantable sound processing module 158 are configured, in at least one embodiment, to execute sound processing logic 190 in memory to convert the received input signals into output signals 195 that are provided to the stimulator unit 142. In such an embodiment, the stimulator unit 142 is configured to utilize the output signals 195 to generate electrical stimulation signals (e.g., current signals) for delivery to the recipient's cochlea, thereby bypassing the absent or defective hair cells that normally transduce acoustic vibrations into neural activity.

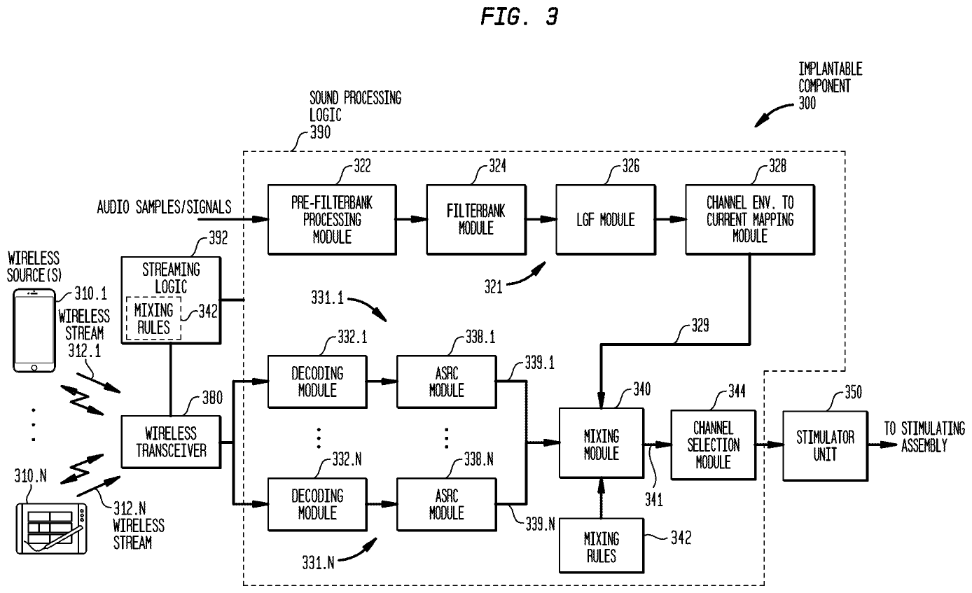

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the implantable component 112 could use signals captured by the sound input devices 118 and the implantable sound sensors 160 in generating stimulation signals for delivery to the recipient. In yet other examples, as discussed in further detail herein below with reference to FIGS. 3-10, an implantable component of a cochlear implant system could use wireless streams received from multiple of wireless sources (via wireless transceiver 180) to generate (via sound processing logic) stimulation signals for delivery to the recipient.

As noted above, implantable medical devices, such as cochlear implant system 102 of FIG. 1D, can include microphones that operate according to operational parameters that allow the microphones to operate with directionality to improve signal-to-noise ratio (“SNR”) of the processed audio signals. This microphone directionality allows recipients to have, for example, improved speech recognition in noisy situations. These microphone directionality techniques rely on the user facing the speaker so the directional microphones can pick up the speaker's voice and block out noise to the sides and rear of listener.

For completeness, it is noted that external sound processing module 124 can be embodied as a BTE sound processing module or an OTE sound processing module. Accordingly, the techniques of the present disclosure are applicable to both BTE and OTE hearing devices.

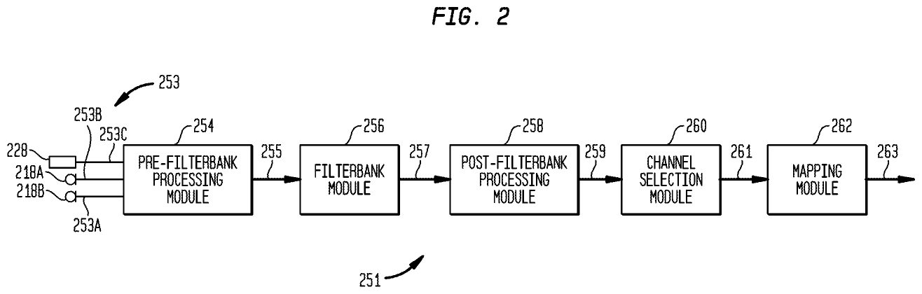

In at least one embodiment during operation of a hearing device system including a cochlear implant, as discussed in further detail below with reference to FIG. 2, sound processing logic 174 is configured to convert output signals received from the input devices 113 (e.g., one or more sound input devices 118 and/or one or more auxiliary input devices 128) into a set of output signals representative of electrical stimulation.

With reference to FIG. 2, shown is a functional block diagram illustrating further details of an example sound/audio signal processing path of an auditory prosthesis, such as cochlear implant system 102, configured to implement certain techniques presented herein. As discussed in further detail with reference to FIGS. 3-10, various sound processing operations discussed for FIG. 2 can be performed via sound processing logic provided for any combination of an external component or an internal component of a cochlear implant system. Various features of cochlear implant system 102 as noted for FIGS. 1A-1D are discussed with reference to various features illustrated in FIG. 2.

Consider, with reference to FIG. 2, a sensory or audio signal processing path 251 which can be provided via sound processing logic 174 of external component 104 and/or via sound processing logic 190 of implantable component 112. In the example of FIG. 2, input devices can include two sound input devices, namely a first microphone 218A and a second microphone 218B, as well as at least one auxiliary input device 228 (e.g., an audio input port, a cable port, a telecoil, etc.). If not already in an electrical form, the input devices can convert received/input sound signals into electrical signals 253, referred to herein as electrical sound or sensory signals, which represent the sound/sensory signals received at the input devices. The electrical sound/sensory signals 253 can include electrical sensory signal 253A from microphone 218A, electrical sensory signal 253B from microphone 218B, and electrical sensory signal 253C from auxiliary input 228.

In FIG. 2, functional operations enabled by the audio signal processing path (i.e., the operations one or more processor(s) when executing sound processing logic) are generally represented by modules 254, 256, 258, 260, and 262 which collectively comprise the audio signal processing path 251. Thus, the audio signal processing path 251 can include a pre-filterbank processing module 254, a filterbank module 256, a post-filterbank processing module 258, a channel selection module 260, and a mapping module 262, each of which are described in greater detail below.

Consider an operational example in which electrical sound signals 253 generated by the input devices are provided to the pre-filterbank processing module 254. The pre-filterbank processing module 254 is configured to, as needed, combine the electrical sound signals 253 received from the input devices and prepare/enhance those signals for subsequent processing. The operations performed by the pre-filterbank processing module 254 can include, for example, microphone directionality operations, noise reduction operations, input mixing/combining operations, input selection/reduction operations, dynamic range control operations and/or other types of signal enhancement operations. The operations at the pre-filterbank processing module 254 generate a pre-filterbank output signal 255 that, as described further below, is the basis of further sound processing operations. The pre-filterbank output signal 255 represents the combination (e.g., mixed, selected, etc.) of the input signals (e.g., mixed, selected, etc.) received at the sound input devices at a given point in time.

In operation, the pre-filterbank output signal 255 generated by the pre-filterbank processing module 254 is provided to the filterbank module 256. The filterbank module 256 generates a suitable set of bandwidth limited channels, or frequency bins, that each includes a spectral component of the received sound/sensory signals. That is, the filterbank module 256 comprises a plurality of band-pass filters that separate the pre-filterbank output signal 255 into multiple components/channels, each one carrying a frequency sub-band of the original signal (i.e., frequency components of the received sound/sensory signal).

The channels created by the filterbank module 256 are sometimes referred to herein as sound processing, or band-pass filtered, channels, and the sound signal components within each of the sound processing channels are sometimes referred to herein as band-pass filtered signals or channelized signals. The band-pass filtered or channelized signals created by the filterbank module 256 are processed (e.g., modified/adjusted) as they pass through the audio signal processing path 251. As such, the band-pass filtered or channelized signals are referred to differently at different stages of the audio signal processing path 251. However, it will be appreciated that reference herein to a band-pass filtered signal or a channelized signal can refer to the spectral component of the received sound signals at any point within the audio signal processing path 251 (e.g., pre-processed, processed, selected, etc.).

At the output of the filterbank module 256, the channelized signals are initially referred to herein as pre-processed signals or filterbank channels 257. The number ‘n’ of filterbank channels 257 generated by the filterbank module 256 can depend on a number of different factors including, but not limited to, implant design, number of active electrodes, coding strategy, and/or recipient preference(s). In certain arrangements, twenty-two (22) channelized signals are created and the audio signal processing path 251 is said to include 22 channels.

The filterbank channels 257 are provided to the post-filterbank processing module 258. The post-filterbank processing module 258 is configured to perform a number of sound processing operations on the target filterbank channels 257. These sound processing operations include, for example, channelized gain adjustments (e.g., performed via Loudness Growth Function (LGF) processing) for hearing loss compensation (e.g., gain adjustments to one or more discrete frequency ranges of the sound signals, also referred to herein as filter channels), noise reduction operations, speech enhancement operations, etc., in one or more of the channels. After performing the sound processing operations, the post-filterbank processing module 258 outputs a plurality of processed channelized signals 259.

In the specific arrangement of FIG. 2, the audio signal processing path 251 includes a channel selection module 260. The channel selection module 260 is configured to perform a channel selection process to select, according to one or more selection rules, which of the ‘n’ channels should be used in hearing compensation. The signals selected at channel selection module 260 are represented in FIG. 2 by arrow 261 and are referred to herein as selected channelized signals or, more simply, selected signals.

In the embodiment of FIG. 2, the channel selection module 460 selects a subset ‘m’ of the ‘n’ processed channelized signals 259 for use in generation of electrical stimulation for delivery to a recipient (i.e., the sound processing channels are reduced from ‘n’ channels to ‘m’ channels). In one specific example, the ‘m’ largest amplitude channels (maxima) from the ‘n’ available combined channel signals are made, with ‘n’ and ‘m’ being programmable during initial fitting, and/or operation of the prosthesis. In one instance, this specific example can be associated with an Advanced Combination Encoder (ACE), generally, a stimulation coding strategy, such as Optimized Pitch and Language (OPAL). It is to be appreciated that different channel selection methods could be used, and are not limited to maxima selection. It is also to be appreciated that, in certain embodiments, the channel selection module 260 can be omitted. For example, certain arrangements can use a continuous interleaved sampling (CIS), CIS-based, or other non-channel selection sound coding strategy.

The audio signal processing path 251 for the instance illustrated in FIG. 2 also includes the mapping module 262, which can generate output signals 263. In one embodiment, the mapping module 262 can be configured to map the amplitudes of the selected signals 261 (or the processed channelized signals 259 in embodiments that do not include channel selection) such that the output signals 263 correspond to a set of stimulation control signals (e.g., stimulation commands) that represent the attributes of the electrical stimulation signals that are to be delivered to a recipient so as to evoke perception of at least a portion of the received sound signals. This channel mapping can include, for example, threshold and comfort level mapping, dynamic range adjustments (e.g., compression), volume adjustments, etc., and can encompass selection of various sequential and/or simultaneous stimulation strategies.

In one embodiment, the set of stimulation control signals (stimulation commands) 263 that represent the electrical stimulation signals can be encoded for transcutaneous transmission (e.g., via an RF link) to an implantable component. As such, mapping module 262 can also be referred to as a channel mapping and encoding module and operates as an output block configured to convert the plurality of channelized signals into a plurality of stimulation control signals, from which the implantable component, via stimulator unit can generate stimulation (current) signals for delivery to the recipient via a stimulating assembly 116.

In one embodiment, for example if channel selection module 260 is omitted from the audio signal processing path 251, the mapping module 262 can perform mapping operations that involve mapping channel envelopes to current levels, which can be mixed with streams received from one or more sources, as discussed in further detail below with reference to FIGS. 3-10 involving various example use cases for implantable components. Generally, a channel envelope is a “temporal envelope” that is extracted from each frequency band (channel) and is used to modulate pulse trains that are delivered to an implanted electrode. Thus, amplitudes of the current pulses can be extracted or mapped from the channel envelopes, where the channel envelopes correspond to the amplitude of the signal in a given frequency channel.

Thus, the audio signal processing path 251 generally operates to convert received sound signals into output signals 153, which can be used for delivering stimulation to a recipient in a manner that evokes perception of the sound signals.

Various signal processing concepts discussed with reference to FIG. 2 will now be discussed with reference to various example use cases for various implantable components, as shown in FIGS. 3-10, in which example implantable components (e.g., of a medical device system, such as a cochlear implant of a hearing device system) can receive, process, and manage wireless streams for any combination of audio/sensory streams and/or stimulation streams that can be received from any combination wireless sources in order to generate/provide stimulation to a recipient of the medical device system.

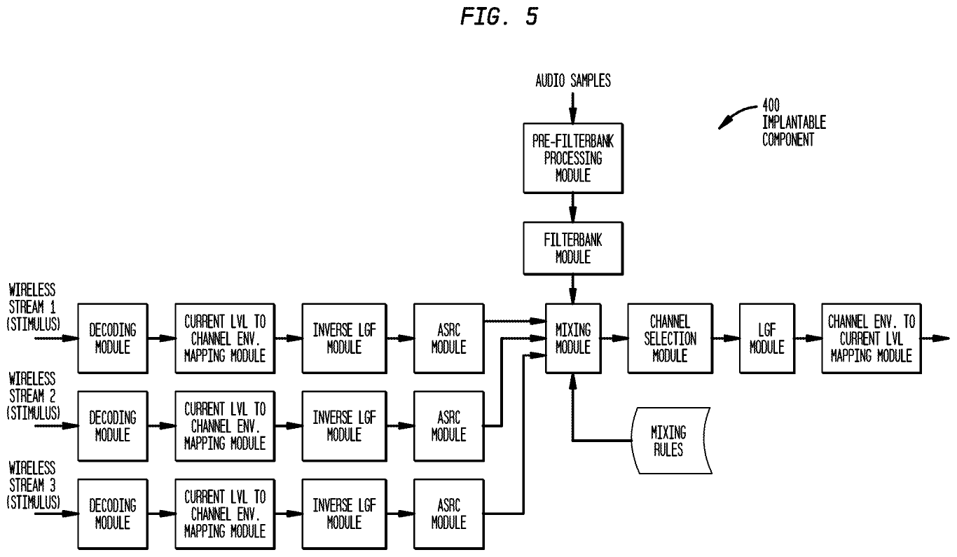

In particular, with reference to FIG. 3, shown is a functional block diagram illustrating details involving an example use case for an implantable component 300 configured to implement techniques presented herein. For the embodiment of FIG. 3, consider that implantable component 300 includes a wireless transceiver 380, a stimulator unit 350, streaming logic 392, and sound processing logic 390 configured with at least one audio signal processing path 321, a number of stimulation stream signal processing paths 331.1-331.N, a mixing module 340, and a channel selection module 344. The embodiment of FIG. 3 illustrates example details in which the implantable component (e.g., cochlear implant) 300 can perform mixing of signal data received from multiple sources in the stimulation signal/data (current level) domain. However, mixing of signal data received from multiple sources can also be performed in the filterbank/channel domain, as discussed in further detail with reference to FIGS. 5-10, below.

Although not shown in FIG. 3, it is to be understood that stimulator unit 350 can generate stimulation signals for a stimulating assembly (not shown) in order to provide stimulation to a recipient of the cochlear implant system.

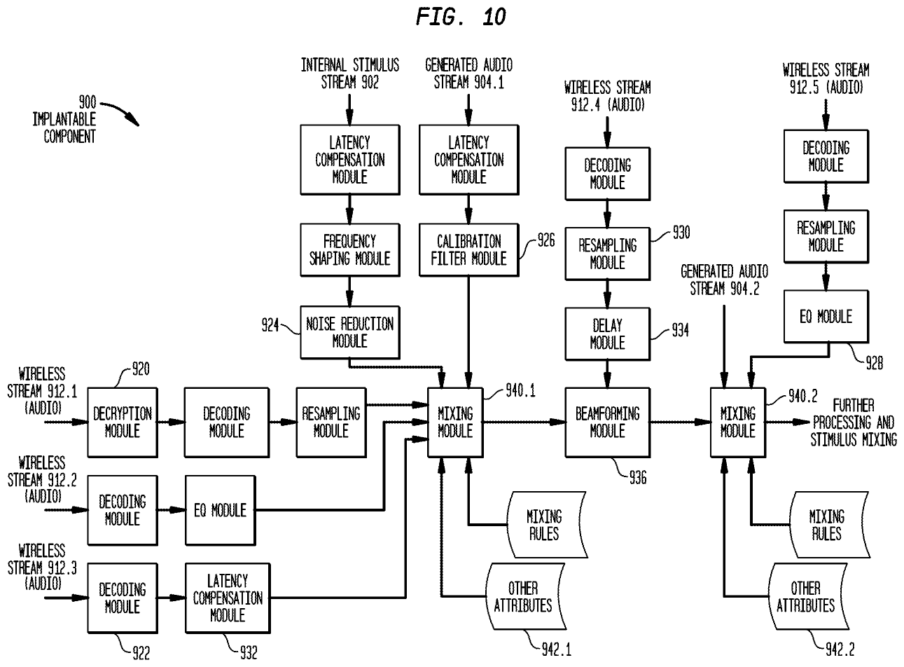

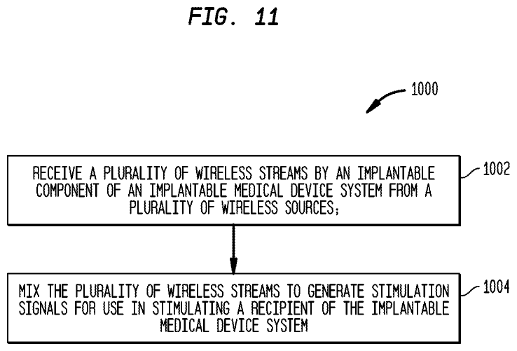

Further, although not illustrated in FIG. 3, it is to be understood that sound processing logic 390 can be configured with one or more audio stream signal processing path(s), which can also generate output signals that can be mixed together with any output signals generated by any combination of one or more audio signal processing paths and/or stimulation stream signal processing paths. Various combinations/types of audio stream signal processing path(s) that can be configured for an implantable component in accordance with embodiments herein are discussed in further detail herein, below, with reference to FIG. 10.

Returning to FIG. 3, also shown are a number of wireless sources 310.1-310.N that can contemporaneously transmit corresponding wireless streams 312.1-312.N to the implantable component 300, which are received via wireless transceiver 380 and processed along corresponding stimulation stream signal processing paths 331.1-331.N. Further, sensory or audio samples/signals (e.g., acoustic sound signals, vibrations, etc.) can be captured via sound sensors 320 and processed along audio signal processing path 321, using signal processing operations similar to those discussed above, with certain variations as discussed in further detail herein, below.

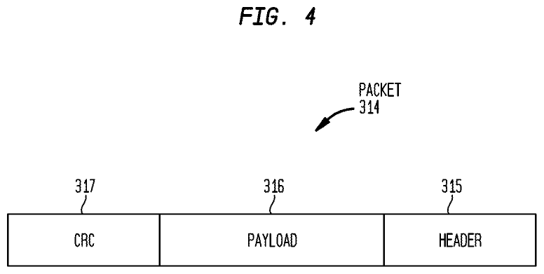

Wireless streams 312.1-312.N contemporaneously received by implantable component 300 can each include a flow of packets, as illustrated in FIG. 4, which is a schematic diagram illustrating one example format for a packet 314 into which signal data (e.g., audio signal data or stimulation signal data), control/management data (e.g., indicating that a wireless source has a stream to be presented to a user/recipient, the type of stream(s), etc.), and/or the like can be encoded/mapped in accordance with embodiments herein (e.g., in a packet sent via a wireless stream). In FIG. 4, the packet 314 comprises a header 315, a payload 316 including signal data, and an error correction field/trailer 317. The payload 316 can include 8 bits, 16 bits, 32 bits, etc. of formatted signal data, control/management data, etc., which can be encoded by a given wireless streaming source (e.g., any of wireless sources 310.1-310.N). The error correction field 317 can include Cyclic Redundancy Check (CRC) information. It is to be appreciated that the size of the 316 can vary from packet to packet for a wireless stream. It is also to be appreciated that the packet format of FIG. 4 is merely illustrative of a packet/frame that can be transmitted by a wireless source and that any wireless source in accordance with embodiments presented herein can use different packet formats to transmit wireless streams.

Returning to the embodiment of FIG. 3, in at least one embodiment, prior to transmitting a wireless stream to the implantable component 300, each wireless source 310.1-310.N can send control/management information (e.g., via one or more packets) to the implantable component 300 for each wireless stream that each wireless source desires to send to the implantable component. In at least one embodiment, the control/management information can include an indication sent to the implantable component 300 indicating that a stimulation signal is to be presented to the recipient via a corresponding wireless stream. In various embodiments, the indication can be a flag, data word, control word, or the like encoded into a data packet/frame transmitted to the implantable component. In some embodiments, each wireless source 310.1-310.N can also indicate a type of each source, which indicates the type of wireless stream, such as a proprietary stimulation protocol type of wireless signal stream/signal data and/or an audio type of wireless signal stream/signal data that is to be provided to the implantable component 300.

In some embodiments, a wireless source can indicate that it can send both types of wireless streams. For example, a simultaneous audio and stimulation encoded data source could be transmitted by a wireless source from multiple inputs or signal paths of the wireless source over the same wireless link in different channels of the same wireless system, such as 2.4/5 GHz Wi-Fi, BLE, or other wireless communication protocol. Thus, in some embodiments, each wireless stream can further include control/management information contained in one or more packet(s) (e.g., in the header of each packet, for the first packet of the stream, etc.) indicating the type of signal data contained in packets of a corresponding stream.

For instances involving a proprietary protocol wireless stream being provided to an implant, a wireless source can further indicate the protocol type and version for the wireless stream, so that the implantable component can correctly decode the received stream. For instances involving regarding audio streaming sources, a wireless source can indicate an encoding type and version for a given audio signal stream so that the implantable component can appropriately decode/decrypt/decompress/etc. the received stream. In some instances, indicating the type of encoding for an audio signal stream may not be needed if implied by the source type from which the stream is obtained.

In various embodiments, streaming logic 392 can receive control/management information from a wireless source and can determine which sources to use (select) and which to ignore for stimulating a recipient. Further, source selection may not be limited to wireless streams. For example, in some embodiments, source selection can involve the implantable component 300 determining whether received audio samples/signals (e.g., received via sound sensors, received from an external component of a cochlear implant (CI) system, etc.) are/are not to be utilized for stimulating a recipient. Thus, streaming logic 390 can interface with sound processing logic 392 to indicate whether one or more streams, as well as audio samples/signals are/are not to be mixed for stimulating a recipient.

Determining when and which sources to select and mix together, as well as how they are mixed together can be achieved by an implantable component (e.g., via streaming logic/sound processing logic) through any combination of techniques involving any combination of selection criteria. For example, in some instances source selection could be based, for example, on recipient (user) preferences (e.g., settings defined and/or adjusted by the recipient/user via an application, device, or the like that can interface with/control/manage a cochlear implant system/implantable component, etc.), ambient environment information, location information, time of day information, predetermined settings (e.g., setting set by a manufacturer/clinician/professional), stream information (e.g., audio content, level, metadata, etc. indicating information of interest (e.g., an incoming phone call can be given priority), stream type, device alarm sounds (e.g., low battery, incoming call, calendar reminder, call waiting, message arrival, email arrival, etc.), environmental alarm sounds (e.g., fire alarm, emergency alert sounds, emergency vehicle sounds, etc.) combinations thereof, and/or the like. In some embodiments, override selections can be defined, in combination with and/or in lieu of mixing sources, such that some sounds may take priority over other sounds, such as device alarm sounds (e.g., low battery, incoming call, calendar reminder, call waiting, message arrival, email arrival, etc.), environmental alarm sounds (e.g., fire alarm, emergency alert sounds, emergency vehicle sounds, etc.). In some embodiments, source selection and mixing rules/techniques can be configured/defined/updated via mixing rules 342 that can be provided for streaming logic 392. Additional examples associated with various techniques through which source signals can be mixed together via mixing module 340 in accordance with various mixing rules 342 that can be configured for implantable component 300 are discussed in further detail below.

Accordingly, streaming logic 392 can interface with wireless transceiver 380 and sound processing logic 390 in order to facilitate using (selecting) or ignoring streams/signals received from different sources, such that wireless transceiver 380 can direct selected streams that are to be used for stimulating the recipient to corresponding stimulation stream signal processing path(s) and/or audio stream signal processing path(s). Further streaming logic 392 can interface with sound processing logic 390 to select whether or not audio samples/signals received/generated/processed via the implantable component 300 and/or an external component (not shown), and, potentially, stimulation signals generated by the external component and/or the implantable component itself are/are not to be mixed together with wireless streams received by the implantable component from one or more sources.

Streaming logic 392 and sound processing logic 390 can also interface based on control/management information provided to the implantable component 300 from a wireless streaming source, such as proprietary stimulation protocol(s), encoding/decoding protocol(s), compression/decompression protocols, encryption/decryption protocols, and/or the like in order to configure corresponding processing to be performed for various signal processing paths provided via the sound processing logic 390, such that streaming signals can be decrypted, decoded, decompressed, interpreted, processed, etc. for combining or mixing together in accordance with embodiments herein. During operation, the implantable component 300, via streaming logic 392, can continuously monitor an environment for new and/or changed source streams and/or source/stream configurations in order to update stimulation provided to a recipient.

In one example for the embodiment of FIG. 3, consider that each corresponding wireless source 310.1-310.N provides an indication that each corresponding source intends to transmit corresponding wireless streams 312.1-312.N of a corresponding proprietary stimulation protocol type, and further the corresponding protocol type and version for each corresponding wireless stream, which triggers stream selection and configuration of sound processing logic 390 to receive and process the corresponding wireless streams 312.1-312.N. It is to be understood that processing operations performed via sound processing logic 390/streaming logic 392 can overlap in any manner.

Broadly, signal processing operations for FIG. 3 can involve generating output signals for each of the audio signal processing path 321 (output signal 329) and the stimulation stream signal processing paths 331.1-331.N (output signals 339.1-339.N) and mixing the output signals together via mixing module 340, utilizing various mixing rules 342 configured for the implantable component 300, as discussed in further detail herein. Thereafter, an output signal 341 generated by the mixing module 340 can be utilized to generate stimulation signals for use in stimulating the recipient via channel selection module 344 and stimulator unit 350.

For example, consider the audio signal processing path 321, which can include a pre-filterbank processing module 322, a filterbank module 324, a Loudness Growth Function (LGF) module 326, and a channel envelope to current mapping module 328. During operation audio samples/signals can be received by the implantable component 300 (e.g., via input devices, such as implantable sound sensors, etc.) and provided to pre-filterbank processing module 322, which can perform any operations as discussed above (e.g., with reference to pre-filterbank processing module 254 of FIG. 2), and signals output therefrom can be provided to filterbank module 324, which can perform any operations as discussed above (e.g., with reference to filterbank module 256 of FIG. 2) to create channelized signals that are provided to LGF module 326. Thereafter, the LGF module 326 applies a loudness growth function and signals generated by the LGF module 326 can be processed via channel envelope to current mapping module 326 to map the channelized signals to output signals 329 representing current levels contained in the channelized signals, which are provided to mixing module 340.

Next, consider the corresponding stimulation stream signal processing paths 331.1-331.N, each of which can include, for the embodiment of FIG. 3, a corresponding decoding module 332.1-332.N, and a corresponding Asynchronous Sample Rate Converter (ASRC) module 338.1-338.N. ASRC is used to convert the sample rate of the incoming wireless streaming signals to the analysis rate of the signal processing in the receiver, as well as to correct the timing drifts between the transmitter and receiver.

During operation for the example illustrated in FIG. 3, the implantable component 300 can be wirelessly paired to any number of wireless sources 310.1-310.N, which can be representative of any combination of external sound processors, mobile phones, TVs, tablets, research devices, and/or audiological streaming devices for objective measures, etc. The incoming wireless streaming signals 312.1-312.N can be encoded as a stimulation data type, which can consist of current levels (CL) and electrode identifiers (IDs), in at least one example. In one example, a particular use case for streaming current levels simultaneously from a number of external devices (streaming sources) could involve a clinician measuring neural responses by sending a known set of current levels to specific electrodes while the recipient is listening to audio activities received via an external and/or implantable sound processor/processing module, which are processed via audio signal processing path 321.

Via corresponding stimulation stream signal processing paths 331.1-331.N, the implantable component 300 decodes the incoming current levels via corresponding decoding modules 332.1-332.2 by using an appropriate decoding method for each stream (e.g., based on control/management information provided by each wireless source 310.1-310.N that is used to configure appropriate decoding methods for decoding modules 332.1-332.N).

As illustrated in FIG. 3, each stimulation stream signal processing path 331.1-331.N can also implement an Asynchronous Sample Rate Converter (ASRC) via corresponding ASRC modules 338.1-338.N for resampling the incoming current levels, if they are arriving at different rates, for example, wireless data packets/frames sent by an objective measurement algorithm could be at a different rate to that of sound related stimuli. The frame rate of decoded current levels from each wireless source 310.1-310.N after processing via corresponding ASRC modules 338.1-338.N can be set to the stimulation rate of the output 329 of the audio signal processing path 321 implantable component sound processing and can then be mixed via mixing module 340, based on one or more mixing rules 342 that can configured/provisioned for the implantable component 300.

Mixing can be performed (e.g., via mixing module 340) using a variety of techniques based on mixing rules 342 that can be configured provisioned for the implantable component 300, in accordance with various embodiments herein. For example, in at least one embodiment mixing rules 342 can include a mixing rule specifying that sources are to be mixed by selecting a maximum current level from the signal data for each source (e.g., stimulation signal data from wireless streaming source(s), audio signal data from wireless streaming source(s), and/or audio signal data from audio samples/signals) for each electrode of a stimulating assembly.

In at least one embodiment, mixing rules 342 can include a mixing rule specifying interleaving signal data generated for each source according to an interleaving scheme. In various embodiments, the interleaving scheme can be set to a round-robin scheme, and alternating scheme, a weighted alternating scheme, or any combination/variation thereof.

In still at least one embodiment, mixing rules 342 can include a mixing rule specifying calculating a weighted sum of current levels contained in the signal data for each source for each of an electrode of a stimulating assembly of the implantable component. A weighting for each source can be set according to a ratio that is desired for each source. The weighting can be set by any combination of a user, a clinician, and/or an automatic weighting.

In still at least one embodiment, mixing rules 342 can include a mixing rule specifying mixing current levels per electrode of a stimulating assembly according to a priority given to the incoming signal type classified by sources themselves or by a classifier in the sound processing logic 392. For example, phone call speech or private alarms could receive a higher priority than another signal types.

In still at least one embodiment, mixing rules 342 can include a mixing rule specifying mixing current levels contained in the signal data for each source based on one or more psychoacoustic masking rules.

In still at least one embodiment, mixing rules 342 can include a mixing rule specifying automatically adjusting a mixing level or frequency response according to a variety of criteria. For example, a mixing rule could specify that loud signal levels can be automatically adjusted using an automatic gain control (AGC) function for each source. In another example, a mixing rule could specify rules for loudness across multiple sources.

In still some embodiments, mixing rules 342 can include any combination or derivation of mixing rules discussed above, which may be relative to mixing stimulation signal/data domain signals, and/or audio/filterbank/channel mixing rules (e.g., for mixing signals in the audio signal domain and/or filterbank/channel domain), as discussed in further detail herein, below, with reference to FIGS. 5-10.

After mixing, other signal processing operations can be utilized by an implantable component prior to generating stimulation for a recipient via a stimulator unit, such as, for example, maxima selection via channel selection module 344, as shown in FIG. 3 (e.g., for ACE, OPAL, or any other stimulation coding strategy), LGF processing, mapping channel envelopes to current levels (e.g., for embodiments in which mixing is performed in the filterbank/channel domain), and/or any cochlear implant signal processing operations that might be utilized in addition to stream decoding, etc., when and where appropriate for the device.

It is to be understood that the example selection criteria for selecting/ignoring sources, as well as the example mixing rules discussed above are provided for illustrative purposes only and are not meant to limit the broad scope of embodiments herein. Any combination of source selection criteria, mixing rules, etc. can be configured for an implantable component in accordance with embodiments herein. Further, it is to be understood that using current levels and electrode IDs as noted for the present example is only one scheme through which sources can be combined or mixed to generate stimulation for a recipient. Other schemes, such as using alternative stimulation levels or magnitude, and channels or groups of electrodes can also be envisioned, including schemes for describing different sequential and/or simultaneous types of stimulation, and, thus, are clearly within the scope of embodiments herein.

Various other example implantable components for cochlear implant systems that can facilitate streaming from multiple wireless sources are illustrated in FIGS. 5-10, in accordance with various embodiments herein.

For example, FIG. 5 is a functional block diagram illustrating details of an example use case for an implantable component 400 configured to implement techniques presented herein. Wireless sources/source devices, a wireless transceiver, streaming logic, and sound processing logic are not shown/labeled in FIG. 5 for purposes of brevity only, in order to discuss other features as illustrated for the implantable component use case of FIG. 5, in which three wireless streams of the proprietary stimulation protocol signal stream source type are shown in FIG. 5 as wireless stream 1, wireless stream 2, and wireless stream 3, which can be contemporaneously received by the implantable component.

Example implantable component 400 for the use case illustrated in FIG. 5 is similar to example implantable component 300 discussed for the use case of FIG. 3 except that decoded current levels from each wireless source are converted to a suitable log-compressed channel envelopes between a minimum level and maximum level and the log-compressed channel envelopes are put through an inverse LGF module so that they can be mixed with the filterbank outputs from the implantable component's own audio signal processing path. In some instances, the implantable component 400 could implement an ASRC module for stimulation stream signal processing paths for resampling the incoming signal data that is received at different frame rates. It is to be understood that placement of the ASRC module could be varied for the stimulation stream signal processing paths. For example, in some instances, ASRC modules could also be placed after the decoder modules for each stimulation stream signal processing path, similar to the processing as illustrated for implantable component 300 of FIG. 3.

Rather than mixing in the stimulation signal/data domain, as shown for the embodiment of FIG. 3, mixing for the embodiment of FIG. 5 can be performed in the filterbank/channel domain. After channel envelopes are mixed, they could then be processed through a typical set of backend CI algorithms such as channel selection, LGF, mapping, and/or any other processing, as can be appropriate for the processing performed by the implantable component.

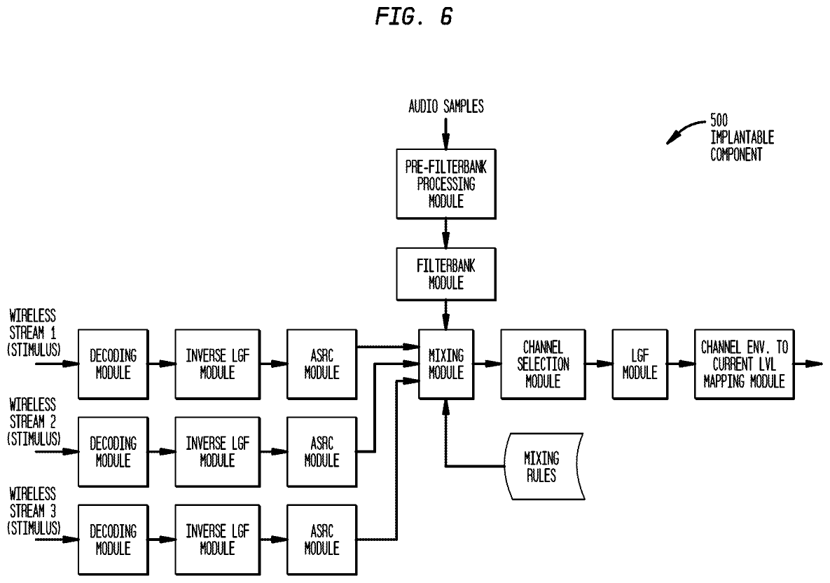

Referring to FIG. 6, FIG. 6 is a functional block diagram illustrating further details of another example use case for implantable component 500 configured to implement certain techniques presented herein. Wireless sources/source devices, a wireless transceiver, streaming logic, and sound processing logic are not shown/labeled in FIG. 6 for purposes of brevity only, in order to discuss other features as illustrated for the implantable component use case of FIG. 6, in which three wireless streams of the proprietary stimulation protocol signal stream source type are shown in FIG. 6 as wireless stream 1, wireless stream 2, and wireless stream 3.

The use case illustrated for the embodiment of FIG. 6 is similar to the use case illustrated for the embodiment of FIG. 6 (mixing in the filterbank/channel domain) except that the decoded levels can already be in a suitable log-compressed channel format between a minimum level and maximum level and, thus, stream signal processing for the embodiment of FIG. 6 can not involve processing to perform log-compressed channel conversion for the signal data processed for each stream.

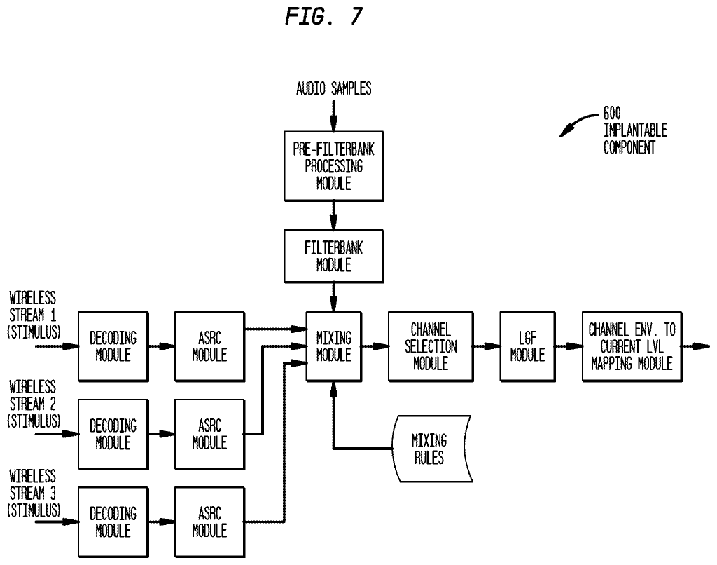

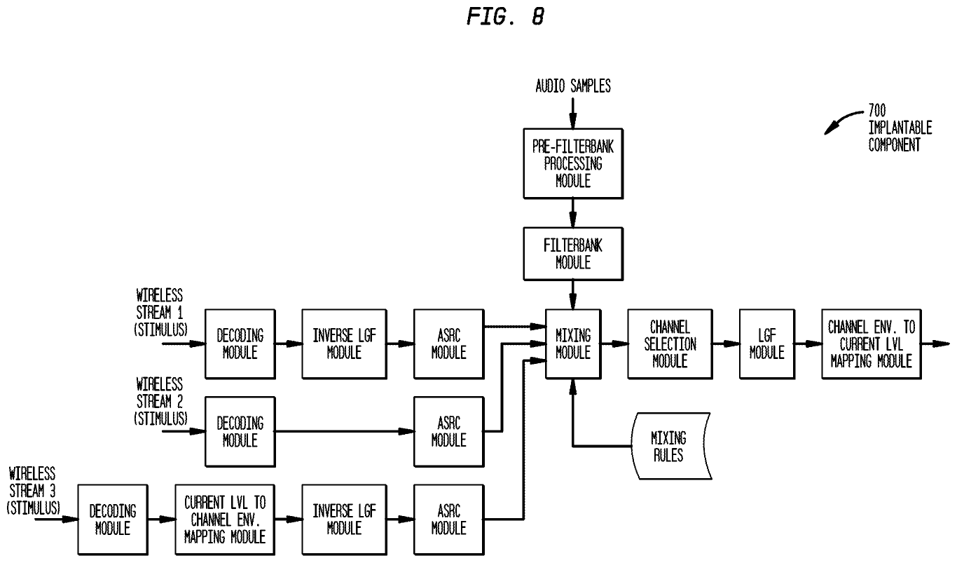

Moving on to FIG. 7, FIG. 7 is a functional block diagram illustrating further details of another example use case for an implantable component 600 configured to implement certain techniques presented herein. Wireless sources/source devices, a wireless transceiver, streaming logic, and sound processing logic are not shown/labeled in FIG. 7 for purposes of brevity only, in order to discuss other features as illustrated for the implantable component use case of FIG. 7, in which three wireless streams of the proprietary stimulation protocol signal stream source type are shown in FIG. 7 as wireless stream 1, wireless stream 2, and wireless stream 3.

The use case illustrated for the embodiment of FIG. 7 is similar to the use case illustrated for the embodiment of FIG. 7 (mixing in the filterbank/channel domain) except that the decoded levels from each wireless source are of the channel envelope type, and are suitable to mix directly with the filterbank outputs of audio signal processing by the implantable component.

Moving to FIG. 8, FIG. 8 is a functional block diagram illustrating further details of another example use case for an implantable component 700 configured to implement certain techniques presented herein. Wireless sources/source devices, a wireless transceiver, streaming logic, and sound processing logic are not shown/labeled in FIG. 8 for purposes of brevity only, in order to discuss other features as illustrated for the implantable component use case of FIG. 8, in which three wireless streams of the proprietary stimulation protocol signal stream source type are shown in FIG. 8 as wireless stream 1, wireless stream 2, and wireless stream 3.

The use case illustrated for the embodiment of FIG. 8 is similar to the use case illustrated for the embodiments of FIGS. 5, 6, and 7 (mixing in the filterbank/channel domain) except that except decoded levels from each wireless source are in different formats. For example, a wireless source sending wireless stream 1 could be sending the stream in an encoded LGF data format, whereas wireless sources sending wireless stream 2 and wireless stream 3 could be sending encoded channel envelopes and encoded current levels for the embodiment of FIG. 8. Depending on the type of incoming signals, the implantable component 700 (via streaming logic/sound processing logic/selection rules/mixing rules) can determine any additional processing that is to be applied to decoded signal data for each stream, before the signal data can be suitably mixed with the filterbank outputs of audio signal processing performed by the implantable component.

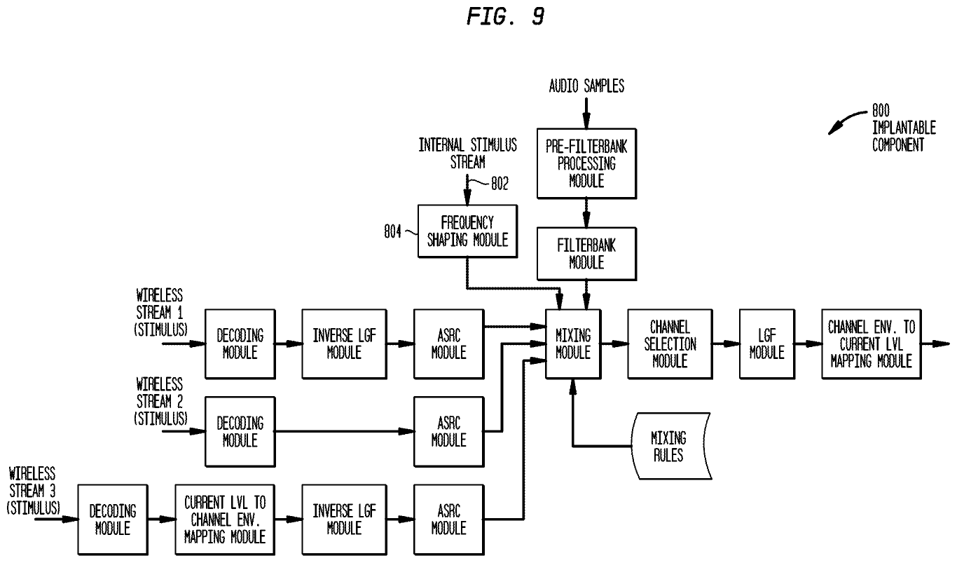

Referring to FIG. 9, FIG. 9 is a functional block diagram illustrating further details of another example use case for an implantable component 800 configured to implement certain techniques presented herein. Wireless sources/source devices, a wireless transceiver, streaming logic, and sound processing logic are not shown/labeled in FIG. 8 for purposes of brevity only, in order to discuss other features as illustrated for the implantable component use case of FIG. 8, in which three wireless streams of the proprietary stimulation protocol signal stream source type are shown in FIG. 9 as wireless stream 1, wireless stream 2, and wireless stream 3.

Further illustrated for the embodiment of FIG. 9 is an internal stimulus stream 802, which could be generated by signal processing performed by implantable component or by an external component of a cochlear implant system. Thus, FIG. 9 illustrates an example use case in which mixing (in the filterbank/channel domain) can be performed with decoded signal data received from wireless sources, the main audio from audio signal processing performed by the implantable component 800 in the channel domain, and also with an internally generated channel domain signal generated via implant processing, such as, for example, noise for tinnitus relief. For example, if internally generated noise signal/sound is for tinnitus relief, a user preference or clinician set frequency profile could be utilized via frequency shaping module 804 that best helps sooth or mask tinnitus. For example, if the user has high frequency tinnitus, then perhaps only the high frequencies of the noise signal/sound may be passed on to the rest of the signal path. The level of the noise could also be user set or clinician set, as part of this processes as well in some embodiments.

Moving to FIG. 10 is a functional block diagram illustrating further details of another example use case for an implantable component 900 configured to implement certain techniques presented herein. Wireless sources/source devices, a wireless transceiver, streaming logic, and sound processing logic are not shown/labeled in FIG. 10 for purposes of brevity only, in order to discuss other features as illustrated for the implantable component use case of FIG. 10, in which various example details are provided to illustrate audio mixing that can be performed by an implantable component, in accordance with embodiments herein.

In accordance with various embodiments herein, audio can be combined to facilitate mixing in the audio signal domain by an implantable component, such as implantable component 900 using numerous techniques, in numerous signal processing places, such as pre/post noise reduction, pre/post beamforming, pre/post filtering/gain operations, etc., with appropriate pre-and post-processing to suit the signal path. Received audio sources, which can be any of encoded audio types (for example in the use of a codec), Pulse-Code Modulation (PCM) audio, or the like. Encrypted audio stream(s), such as wireless stream 912.1, shown in FIG. 10, can be first processed such that certain properties of the audio stream(s) match prior to mixing. Received audio streams can be optionally decrypted and decoded to be of the same audio format suitable for mixing.

Various audio sources that can be mixed by an implantable component can include any combination of: audio streams (one or more channels) from internal microphones on the implant (such as 902, shown in FIG. 10); audio streams generated internally (such as 904.1 and 904.2, shown in FIG. 10), which can include test signals, audiometric signals used by a clinician or other audio (e.g., to perform clinical testing, objective measures (measuring neural responses), private alarm sounds (to indicate a low battery, for example), noise (e.g., for tinnitus masking), and/or the like; wireless audio streams (one or more channels) received from those decoded levels an ipsilateral sound processor or hearing device, received from a contralateral sound processor or hearing device, received from an external microphone, received from a wireless accessory (computer, tablet, mobile phone, TV, Roger FM or other dedicated accessories), received from a telecoil or broadcasting device, received from a network or internet connection (implant connected to Wi-Fi for example) (such as any of wireless streams 912.1-912.5, shown in FIG. 10), inferred audio received from an accelerometer or other sensors that are either internal or external to the implantable component 900, and/or the like.

Received audio streams can be optionally pre-processed prior to mixing using any combination of processing such as decryption (performed, for example, via an appropriately configured decryption module 920, as shown in FIG. 10 (e.g., configured based on control/management information indicating encryption/decryption type, format, security keys, etc. received from a wireless source), for secure audio sources/communications), decoding (performed for example, via an appropriately configured decoding module 922, as shown in FIG. 10 (e.g., configured based on control/management information indicating codec information, audio format, etc. received from a wireless source), for use of codecs and audio formats for low bandwidth audio), noise reduction algorithms (e.g., an SNR-based noise reduction system performed, for example, via an appropriately configured noise reduction module 924, as shown in FIG. 10), filtering for the purpose of matching frequency response and/or phase response (e.g., Finite Impulse Response (FIR) equalization filtering or Infinite Impulse Response (IIR) filtering performed, for example, via an appropriately configured calibration filtering module 926, as shown in FIG. 10 in order to match the frequency and phase response of a stream of audio to the frequency and phase characteristics of another stream of audio, such that the two streams can be mixed together and have a desired response), filtering for frequency emphasis (e.g., applying an equalizer (EQ) function performed, for example, via an appropriately configured EQ module 928, as shown in FIG. 10), re-sampling with appropriate anti-aliasing filters to match sampling frequencies (performed, for example, via an appropriately configured resampling module 930, as shown in FIG. 10), clock rate synchronization to adjust audio to account for clock drift between various received sources (which can, for example, be combined with a re-sampler that allows for sub-sample adjustment and/or processing to drop/interpolate samples), latency compensation to synchronize temporal information from various sources and/or achieve certain desired delays by use case or user preference (performed, for example, via any appropriately configured latency compensation module 932 and/or appropriately configured delay module 934, as shown in FIG. 10.

Audio can be mixed in using various techniques and/or mixing processes, which can, in some embodiments be performed via multiple mixing stages, shown in FIGS. 9 as 940.1 and 940.2. For example, in at least one embodiment, straight mixing can be performed as: \vec{y₀}=\vec{x₁}+\vec{x₂}+ . . . +\vec{xₙ} in which an ‘n’ number of audio signals (signal data) can be added together. In at least one embodiment, mixing static gain mixing can be performed as: \vec{y₀}=g₁\vec{x₁}+g₂\vec{x₂}+ . . . +gₙ\vec{xₙ} in which an ‘n’ number audio signals can have a corresponding gain (gₙ) applied thereto (e.g., clinically/user/device set), per-audio stream, prior to mixing. Such gains have sometimes been described as “mixing ratios.”

In at least one embodiment, dynamic gain mixing can be performed as: \vec{y₀}=g₁(\vec{x₁}, \vec{a₁})\vec{x₂}+g₂(\vec{x₂}, \vec{a₂})\vec{x₂}+ . . . +gₙ(\vec{xₙ}, \vec{aₙ})\vec{xₙ} or \vec{y₀}=g₁(\vec{x₁}, \vec{x₂}, . . . , \vec{xₙ}, a\vec{a₁})\vec{x₁}+g₂(\vec{x₁}, \vec{x₂}, . . . , \vec{xₙ}, \vec{a₂})\vec{x₂}+ . . . +gₙ(\vec{x₁}, \vec{x₂}, . . . , \vec{xₙ}, \vec{aₙ})\vec{xₙ} in which the gain to be used is dynamic, dependent on any combination of static gains (e.g., clinically/user/device set), audio signal characteristics of both an audio stream (e.g. broadband level for per-stream AGC) and other audio streams, and other attributes (942.1 and 942.2, as shown in FIG. 10, per mixing stage) that can be relevant in determining the appropriate gains (e.g., scene/environment classification, etc.).

In at least one embodiment, filtered mixing can be performed as: \vec{y₀}=g₁(\vec{x₁}, \vec{x₂}, . . . , \vec{xₙ}, \vec{a₁})f(\vec{x₁})+g₂(\vec{x₁}, \vec{x₂}, . . . , \vec{xₙ}, \vec{a₂})f(\vec{x₂})+ . . . +gₙ(\vec{x₁}, \vec{x₂}, . . . , \vec{xₙ}, \vec{aₙ})f(\vec{xₙ}) in which mixed audio can be a combined function of dynamic gains and filtering (linear and/or non-linear (f(\vec{xₙ})) operations. Similar to the gain determination, a given filter can either be static (pre-determined, or set by clinicians/users, for example, an EQ on a music source) or dynamic (depending on the signal characteristics alone and/or in combination with other audio sources). Another application of this type of mixing is for use in beamformers (performed, for example, via an appropriately configured beamforming module 936, as shown in FIG. 10) in which one or more audio sources can have a fractional-delay filter applied thereto, before addition/subtraction of the signals. In some instances, this can potentially by used by an implantable component that has two audio sources streamed from two or more external devices, where the audio sources are combined to produce a beamforming pattern with desired directionality. Another application of this type of mixing can be used in noise reduction algorithms where, for example, one audio source can be a signal of interest, and another can be a noise reference signal. In such an application, the two audio sources can be “mixed” to reduce the perception of noise in the signal of interest.

It is to be understood that any combination of the above forms of mixing can be performed by an implantable component, in accordance with embodiments herein. Further, the above mixing examples are only provided for illustrative purposes and are not meant to limit the broad scope of embodiments herein. Virtually any forms of mixing and/or combination of mixing, which may be derived, enhanced, etc. from examples discussed herein, can be performed by an implantable component and, thus, is clearly within the scope of embodiments herein.

With reference now made to FIG. 11, depicted therein is a flowchart of a method 1000 for implementing the techniques of the present disclosure. Method 1000 begins in operation at 1002, which can include receiving a plurality of wireless streams by an implantable component of an implantable medical device system from a plurality of wireless sources. At 1002, the method includes mixing the plurality of wireless streams to generate stimulation signals for use in stimulating a recipient of the implantable medical device system. As discussed for various embodiments herein, the mixing can include selecting and mixing certain streams in order to generate the stimulation signals. Accordingly, the method of flowchart 1000 provides for a process in which multiple streams can be received by an implantable component of a medical device system and used to stimulate a recipient of the medical device system.

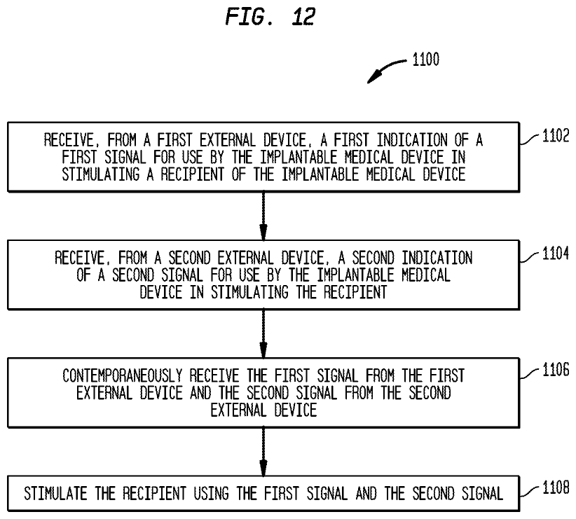

With reference now made to FIG. 12, depicted therein is a flowchart of a method 1100 for implementing the techniques of the present disclosure. Method 1100 begins in operation at 1102, which can include receiving, from a first external device, a first indication of a first signal for use by the implantable medical device to generate stimulation signals for delivery to a recipient of the implantable medical device. At 1104, the method can include receiving, from a second external device, a second indication of a second signal for use by the implantable medical device to generate stimulation signals for delivery to the recipient. At 1106, the method can further include contemporaneously receiving the first signal from the first external device and the second signal from the second external device. At 1108, the method can include generating stimulation signals for delivery to the recipient based on the first signal and the second signal.

Accordingly, the method of flowchart 1100 provides for a process in which multiple streams can be received by an implantable component of a medical device system and used to stimulate a recipient of the medical device system

Merely for ease of description, the techniques presented herein have primarily described herein with reference to illustrative medical device systems, namely cochlear implant hearing device systems, which can deliver any combination of electrical stimulation to a recipient. However, it is to be appreciated that the techniques presented herein can also be used with a variety of other medical devices that, while providing a wide range of therapeutic benefits to recipients, patients, or other users, can benefit from the techniques presented.

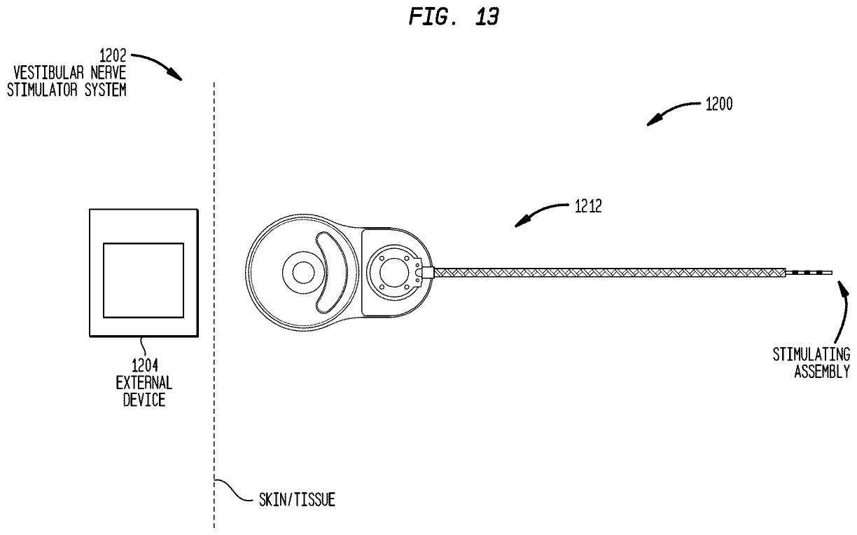

Furthermore, it is to be appreciated that the techniques presented herein can be used with other systems including two or more devices, such as systems including one or more personal sound amplification products (PSAPs), one or more acoustic hearing aids, one or more bone conduction devices, one or more middle ear auditory prostheses, one or more direct acoustic stimulators, one or more other electrically simulating auditory prostheses (e.g., auditory brain stimulators), one or more vestibular devices (e.g., vestibular implants), one or more visual devices (i.e., bionic eyes), one or more sensors, one or more pacemakers, one or more drug delivery systems, one or more defibrillators, one or more functional electrical stimulation devices, one or more catheters, one or more seizure devices (e.g., devices for monitoring and/or treating epileptic events), one or more sleep apnea devices, one or more electroporation devices, one or more remote microphone devices, one or more consumer electronic devices, etc. For example, FIG. 13 is schematic diagrams of alternative systems that can implement aspects of the techniques presented herein.

More specifically, FIG. 13 is a schematic diagram illustrating an example vestibular system 1200 that can be configured to perform synchronized spectral analysis, in accordance with certain embodiments presented herein. In this example, the vestibular system 1200 comprises a vestibular stimulator 1202. The vestibular stimulator 1202 comprises an external device 1204 and an implantable component 1212. In accordance with certain embodiments presented herein, the vestibular stimulator 1202 (e.g., external device 1204 and/or implantable component 1212) are configured to implement aspects of the techniques presented herein to perform multi-band channel coordination of received/input signals (e.g., audio signals, sensor signals, etc.), in accordance with various embodiments herein.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1. A method comprising:
receiving a plurality of wireless streams by an implantable component of an implantable medical device system from a plurality of wireless sources; and
mixing the plurality of wireless streams to generate stimulation signals for use in stimulating a recipient of the implantable medical device system.

2. The method of claim 1, wherein the plurality of wireless streams include a plurality of wireless stimulation signal streams.

3. The method of claim 1, wherein the plurality of wireless streams include a plurality of wireless audio signal streams.

4. The method of claim 1, wherein the mixing comprises:
converting each wireless stream of the plurality of wireless streams to signal data for each wireless stream; and
mixing the signal data for each wireless stream based on one or more mixing rules configured for the implantable component to generate the stimulation signals for use in stimulating the recipient of the implantable medical device system.

5. The method of claim 4, wherein signal data is mixed in at least one of a stimulation signal domain, a channel domain, or an audio signal domain.

6. The method of claim 4, wherein the converting includes decoding each wireless stream of the plurality of wireless streams to generate the signal data for each wireless stream.

7. The method of claim 6, wherein the signal data for each wireless stream comprises current levels corresponding to stimulation signals associated with each wireless stream for stimulating electrodes of a stimulating assembly of the implantable component.

8. The method of claim 4, wherein the converting includes one or more of:
mapping current levels contained in the signal data for at least one wireless stream to log-compressed channel envelopes between a minimum level and a maximum level for at least one wireless stream;
performing inverse loudness growth function processing on the signal data for at least one wireless stream to remove gain included in the signal data;
resampling the signal data for at least one wireless stream;
performing latency compensation for the signal data for at least one wireless stream;
performing frequency shaping for the signal data for at least one wireless stream; or performing noise reduction for the signal data for at least one wireless stream.

9. The method of claim 4, wherein the one or more mixing rules include one or more of:
a mixing rule indicating that the plurality of wireless streams are to be mixed by selecting a maximum current level from the signal data generated for each wireless stream for each of an electrode of a stimulating assembly of the implantable component;
a mixing rule indicating interleaving signal data generated for each wireless stream;
a mixing rule indicating calculating a weighted sum of current levels contained in the signal data for each wireless stream for each of an electrode of a stimulating assembly of the implantable component;
a mixing rule indicating mixing current levels for each of an electrode of a stimulating assembly of the implantable component based on a priority corresponding to a type of each wireless stream; or
a mixing rule indicating mixing current levels contained in the signal data for each wireless stream based on one or more psychoacoustic masking rules.

10. The method of claim 4, wherein the mixing comprises mixing signal data for audio received by the implantable component along with the signal data for each wireless stream based on the one or more mixing rules configured for the implantable component to generate the stimulation signals for use in stimulating the recipient of the implantable medical device system.

11. The method of claim 4, wherein the mixing comprises:
mixing the signal data for each wireless stream based on the one or more mixing rules to generate first mixed output signals;
performing one or more signal processing operations of the first mixed output signals to generate processed signals; and
mixing the processed signals with one or more wireless streams to generate the stimulation signals for use in stimulating the recipient of the implantable medical device system.

12-23. (canceled)

24. One or more non-transitory computer readable storage media comprising instructions that, when executed by a processor, cause the processor to:
receive, from a first external device, a first indication of a first signal for use by a implantable medical device in stimulating a recipient of the implantable medical device;
receive, from a second external device, a second indication of a second signal for use by the implantable medical device in stimulating the recipient;
contemporaneously receive the first signal from the first external device and the second signal from the second external device; and
stimulate the recipient using the first signal and the second signal.

25. The one or more non-transitory computer readable storage media of claim 24, wherein the first indication and the second indication indicate a type of the first signal and the second signal.

26. The one or more non-transitory computer readable storage media of claim 25, wherein the type indicates one of a proprietary stimulation protocol signal or an audio signal.

27. The one or more non-transitory computer readable storage media of claim 26, further comprising instructions that, when executed by a processor, cause the processor to:
receive from at least one of the first external device or the second external device, one or more of, protocol type information, protocol version information, an encoding type indication, or encryption information for at least one of the first signal or the second signal.

28. The one or more non-transitory computer readable storage media of claim 24, further comprising instructions that, when executed by a processor, cause the processor to:
receive from a third external device, a third indication of a third signal for use by the implantable medical device in stimulating the recipient; and
contemporaneously receive the first signal from the first external device, the second signal from the second external device, and the third signal from the third external device.

29. The one or more non-transitory computer readable storage media of claim 28, wherein to stimulate the recipient includes stimulating the recipient using the first signal and the third signal and ignoring the second signal.

30. The one or more non-transitory computer readable storage media of claim 29, wherein ignoring the second signal is based on one or more of a user setting, a location, a time of day, or a type of the second signal.

31. The one or more non-transitory computer readable storage media of claim 24, wherein to stimulate the recipient using the first signal and the second signal includes combining the first signal and the second signal to generate a stimulation signal for stimulating the recipient.

32. The one or more non-transitory computer readable storage media of claim 24, wherein the first signal and the second signal are received by the implantable medical device via one or more wireless communication protocols.

33. The one or more non-transitory computer readable storage media of claim 32, wherein the first signal and the second signal are received by the implantable medical device via different channels of a same wireless communication protocol.

34. The one or more non-transitory computer readable storage media of claim 24, wherein at least one of the first signal or the second signal is an audio signal.

35-46. (canceled)

47. An implantable component of an implantable medical device system comprising:
memory for storing data; and
one or more processors, wherein the one or more processors are configured to:
receive a plurality of wireless streams from a plurality of wireless sources; and
mix the plurality of wireless streams to generate stimulation signals for use in stimulating a recipient of the implantable medical device system.

48. The implantable component of claim 47, wherein the plurality of wireless streams include a plurality of wireless stimulation signal streams.

49. The implantable component of claim 47, wherein the plurality of wireless streams include a plurality of wireless audio signal streams.

50. The implantable component of claim 47, wherein to mix the plurality of wireless streams, the one or more processors are further configured to:
convert each wireless stream of the plurality of wireless streams to signal data for each wireless stream; and
mix the signal data for each wireless stream based on one or more mixing rules configured for the implantable component to generate the stimulation signals for use in stimulating the recipient of the implantable medical device system.

51. The implantable component of claim 50, wherein signal data is mixed in at least one of a stimulation signal domain, a channel domain, or an audio signal domain.
